(19) United States
(12) Patent Application Publication
(10) Pub. No.: US 2021/0308033 A1
(43) Pub. Date: Oct. 7, 2021

(54) CHLORINE-MODERATING LIQUID COMPOSITIONS FOR USE ON THE HUMAN BODY AND HAIR

(71) Applicant: Jonathan G. Foss, Waconia, MN (US)

(72) Inventor: Jonathan G. Foss, Waconia, MN (US)

(21) Appl. No.: 17/345,206

(22) Filed: Jun. 11, 2021

Related U.S. Application Data
(63) Continuation-in-part of application No. 16/720,938, filed on Dec. 19, 2019.

(51) Int. Cl. A61K 8/67; A61K 8/02; A61K 8/19; A61Q 17/00; A61Q 19/10; A61Q 5/02

(52) U.S. Cl. CPC A61K 8/676; A61K 8/0208; A61Q 5/02; A61Q 17/00; A61Q 19/10; A61K 8/19

(57) ABSTRACT

A method of dechlorinating skin, especially dechlorinating skin, hair and/or nails of humans including contacting said skin, hair and/or nails with an aqueous composition for sufficient time to reduce halogen effects on the skin, hair and/or nails. The aqueous composition includes at least water and an oil-soluble bis-ascorbate in a weight ratio of about 0.1 to 25% by weight of the total aqueous composition and ).05% to 30% by weight of suspended carbon particles. The composition may be free of ascorbic acid, and the oil-soluble ascorbate is a bis or tri- or tetraalkanol ascorbate such as bisglyceryl ascorbate.

### RELATED APPLICATION DATA SECTION

This application claims priority under 35 U.S.C. 120 as a continuation-in-part application of U.S. patent application Ser. No. 16/720,938, filed, 19 Dec. 2020.

### BACKGROUND OF THE INVENTION

### 1. Field of the Invention

The disclosure relates to compositions and methods for decreasing or eliminating adverse effects of chlorine on the human body, including the skin and hair.

### BACKGROUND OF THE ART

Humans come into contact with chlorine in a number of different ways. For example, swimming is a popular form of exercise and pastime. By its nature, swimming requires immersing oneself in a body of water. People may swim in either natural bodies of water (such as lakes, oceans, rivers, etc.), or man-made swimming pools.

Man-made swimming pools (including traditional swimming pools, hot tubs, municipal water supplies, etc., collectively referred to generally herein as “pools”) are usually smaller than naturally occurring bodies of water. They are also usually self-contained structures, consisting of a finite body of water separated from the surrounding environment, for example by walls. Pools provide an aqueous environment that is kept within a biologically habitable temperature range, such as about 65-90° F. with a Free Chlorine residual between 1 and 5 ppm and a pH between 7.2 and 7.8. Some pools may be kept cooler or warmer. For example, a hot tub may be maintained at more than 95° F. but typically use bromine as Hypochlorous Acid is increasingly ineffective at higher temperatures.

A swimmer brings a variety of living and non-living substances into the pool. For example, the swimmer's skin, hair, saliva, urine, sweat, and other secretions may come into contact with the pool water. Owing to the aqueous medium and adequate temperature, pools provide a suitable environment for living organisms, such as bacteria, to thrive.

Pools are usually treated with oxidizing chemicals chosen to prevent the growth of harmful organisms, such as bacteria and parasites like E. coli and cryptosporidium. Properly used, these chemicals keep the pool water substantially free from harmful contaminants. For example, many pools are treated with oxidizing agents (e.g., chlorine, hypochlorite salts such as calcium hypochlorite or sodium hypochlorite, hypochlorous acid) or to a much lesser extent, brominating or iodating agents (e.g., bromine, salts comprising bromine, iodine, etc.). These Halogens sanitize the water at low levels and serve as oxidizers at higher levels.

The chemicals used to treat pool water work by reacting with certain molecules that come into contact with the pool water. For example, these chemicals may react with bacteria's biologically important molecules, thereby killing the bacteria.

In addition to reacting with bacteria and other contaminants, however, some pool chemicals react with elements of the swimmer, such as the swimmer's body, the swimmers' hair and/or the swimmer's attire. For example, the fibers that make up the exterior of the body, such as skin, hair, eyes, and nails (collectively referred to herein as “exterior body elements” of “body fibers,” and intending to include keratinous fibers that make up the hair, skin, and nails, as well as mucous membranes), comprise proteins. By way of example, human hair is made largely from alpha keratin (alpha-keratin). Those proteins are made from amino acids. All amino acids, including those making up proteins, have one or more N—H bonds. In alpha-keratin, the most abundant amino acid is cystine, which accounts for about 15% of the protein. Monomeric L-cystine has two N—H bonds. The oxidized dimmer of cystine has four N—H bonds. When present within a protein, each cystine residue has one N—H bond.

The N—H bonds in the amino acids in body fibers can react with the chemicals found in pool water. For example, one or more N—H bonds in an amino acid in the protein of hair or skin can react with a chlorinating agent used in pool water to form N—Cl, an amino chloride. Notably, the reacted amino acid, now containing an N—Cl bond, is still part of the protein in the hair or skin.

Although a swimmer can rinse the residual pool water from his/her skin, hair, attire, etc., after swimming, such rinsing will not effectively eliminate all the adverse effects of exposure to the chemically-treated pool water. Traditional soaps are ineffective at breaking the millions of N-CL bonds that form on the skin and hair. For example, where the body's proteins have chemically reacted with the pool chemicals, they are physically changed but, at least in part, remain part of the body, i.e., they are not all rinsed away such as during normal showering.

Those remaining pool chemicals can be released throughout the day, for example as a result of exposing the skin, hair, etc., to moisture, e.g., water. As discussed above, after swimming in a pool with a chlorinating and/or brominating agent, proteins of the human body may become chlorinated and/or brominated. Subsequent exposure of those chlorinated and/or brominated amino groups to water (e.g. rain or sweat) may release these volatile chemicals, which may be corrosive or irritating. Certain skin conditioning agents have reacted with chlorine chemicals on the skin and can even cause extreme reactions. One skin moisturizer when applied to dry skin after swimming in a chlorinated pool reacted badly, turning the skin of the swimmer “red as a lobster” and caused intense burning pain as it reacted with the chlorine compounds. The particular conditioner plus dried Hypochlorous acid N-CL bonds didn't mix well. The previous composition was used and repeated this experiment many times, just in very small areas, and skin always reacted the same.

Some of these corrosive molecules may be harmful to body and/or textile fibers (e.g. clothing such as swimming attire), or may cause an unpleasant sensation upon contact with the body and/or clothing fibers. Additionally, some of the volatile molecules may be perceived by the nose when liberated from the body and/or clothing, giving rise to odors. These odors are commonly referred to as simply “chlorine” or “pool odor,” and are considered a more chemical odor, rather than the type of odor naturally produced by the body. Swimmers post workout can be smelled (wreaking) From 12 feet away. Chorine is odorless, but incredibly pungent as a mono- di- or tri-chloramine.

Also, when pool chemicals, such as chlorine or bromine, react with the biological molecules forming the skin and/or eyes, those reactions may cause irritation. For example, some swimmers report itchy or inflamed skin following swimming in pools. Some swimmers indicate that mucous membranes, such as the sensitive nasal skin, become itchy and irritated following swimming.

Although some people have reported liking “pool odor,” as reminiscent of the pleasures associated with swimming itself, most people do not like pool odor or, if strong enough, find the odor irritating, such as to the eyes and lungs. Additionally, since the chemicals liberated may irritate the skin and/or damage the hair, many people wish to prevent “pool odor” and/or the symptoms associated with it. Additionally, green hair, shiny hair and loss of body hair are secondary problems associated with the N-CL reaction with human skin and hair.

As discussed above, rinsing or washing, e.g. body fibers, does little to eliminate pool odor and/or skin irritation. Mitigating (i.e. decreasing to some extent or eliminating entirely) the effects of exposure to chlorinating and/or brominating agents requires reversing the chemical reaction between those chemicals and the proteins making up the human body. This requires converting the amino-halide bonds, e.g., chloramine (N—Cl) and/or bromamine (N—Br) groups into amino (N—H) groups. However, the soaps, shampoos, and conditioners currently known do not effectively convert N—Cl and/or N—Br bonds on body fibers back into N—H bonds. Accordingly, N—Cl and/or N—Br remain bonded to the body fibers following rinsing, washing, shampooing, and/or conditioning the body fibers, such as the skin and/or hair.

Some known shampoo and soap formulations are directed to mitigating the effects of exposure to chlorinating and/or brominating agents. For example, U.S. Pat. No. 4,295,985 discloses “a method of removal of chlorine retained by human skin and hair after exposure to chlorinated water, and soap and shampoo compositions adapted to effect said removal.” That patent teaches applying urea and thiosulfate salts to the hair and/or body following exposure to chlorinating agents.

Other known formulations have sought to remove minerals from hair in an effort to prevent discoloration of the hair. For example, U.S. Pat. No. 5,804,172 discloses compositions aimed at removing mineral deposits from hair exposed to hard water, particularly the calcium, magnesium, iron, and copper present in some municipal water sources. That patent discloses compositions including four ingredients, which are said to remove minerals from the hair due to the “synergistic combination” of ingredients. Within those compositions, a reducing agent, such as ascorbic acid, is included in an amount chosen to reduce oxidized cysteine-iron bonds. The patent discloses four-component compositions comprising 2.1 percent w/w of ascorbic acid, which is said to be sufficient to reduce the oxidation state of iron ions bonded to hair.

Additional known formulations have sought to remove chlorine from hair by treating the hair with ammonium lauryl sulfate, cocamide diethanolamine, sodium bicarbonate, cocobetaine, and water. See U.S. Pat. No. 4,547,364.

Finally, a host of other formulations promise to treat damaged hair and/or skin following exposure to swimming pools by using various combinations of ingredients. For example, U.S. Pat. No. 4,690,818 discloses a combination of hair and skin conditioners and moisturizers, namely, “a combination of coco-diammonium hydrolyzed keratin and a mixture of monosaccharides and disaccharides . . . .”

U.S. Pat. No. 9,302,909 (Chadeayne) discloses methods of mitigating the effects of exposure to oxidizing agents, chlorinating and/or brominating agents on biological and/or synthetic fibers, by treating the body and/or clothing fibers with an effective amount of a composition comprising compounds of formula A, which include ascorbic acid and sodium salts of ascorbic acid (sodium ascorbate). Also disclosed are compositions comprising compounds of formula A, for use in treating biological and/or synthetic fibers to mitigate the effects of exposure to chlorinating and/or brominating agents.

U.S. Pat. No. 4,295,985 discloses a method of removal of chlorine retained by human skin and hair after exposure to chlorinated water, and soap and shampoo compositions adapted to effect said removal by removing rapidly chlorine retained by the skin and hair of, for example, swimmers, after exposure to chlorinated water by means of a stoichiometric excess of a suitable reducing agent, such as an alkali thiosulfate and the like in aqueous solution, and with toilet soaps and shampoos comprising said agent adapted to effect said removal.

U.S. Pat. No. 7,244,453 (Lucia M. Litman) discloses that retention of chlorine and hypochlorite in hair is reduced by treating the hair with a defined composition, allowing the composition to remain on the hair for at least 10 seconds, and then rinsing off the composition with water. The composition comprises at least 1% by total weight of the composition of potato particles having an average diameter of less than 10 microns when dried; at least 1% by total weight of the composition of clathrate; at least 1% by weight of surfactant; and water.

Carbon particles have long been used as absorbent, adsorbents and filtration materials to remove other materials polluting water, are and other liquids. Typically the particles are secured within a porous layer or provided as a bed of particles through which fluids (gas or liquids) are passed to absorb contaminants. Examples of these are in the following U.S. Patents.

U.S. Pat. No. 4,248,705 (Vadekar) evidences an improved process for removing oil from oily waste water streams, particularly from aqueous refinery streams wherein the oil is present as a stable oil-in-water emulsion, which comprises passing the stream through a filter bed of particulate, unprocessed PVC or its copolymers mixed with a granular, inert and preferably porous filler material such as particles of coke or carbon. The improvement substantially increases bed life and has unexpectedly resulted in higher oil-absorption capacity of the filter bed.

U.S. Pat. No. 4,238,334 (Halbfoster) evidences a method and filter bed for removing impurities from liquids wherein the filter bed comprises a mixture of treated filter aid material and an active particulate material having opposite surface charges in aqueous suspension. The filter aid material has been treated to produce the desired surface charge. The identity of the active particulate material depends on the application. Examples are organic polymeric absorbents, zeolites, bentonite, zirconium oxide, zirconium phosphate, activated alumina, ferrous sulfide, activated carbon and diatomaceous earth. s Activated carbon is made by forming a slurry of carbonaceous and non-carbonaceous fine particles, preferably finely divided non-caking coal, in a suspension liquid, preferably water, comprised of at least 60 percent suspension liquid by weight. Preferably, the geometric mean particle size of the fine particles is less than 150 microns. A free-flowing liquid, which is liquid below 80 C. and preferably below 20 C, lyophobic to the suspension liquid and the non-carbonaceous fine particles and lyophilic to the carbonaceous fine particles, is added to the slurry to form a mixture where less than 20 percent by weight of the free-flowing liquid and the carbonaceous and non-carbonaceous fine particles is free-flowing liquid. The resulting mixture is then agitated to preferentially agglomerate the carbonaceous fine particles to form discrete agglomerates, while the non-carbonaceous fine particles remain substantially unagglomerated in the slurry. The discrete agglomerates of carbonaceous material are thereafter separated from the mixture, and carbonized by heating to greater than about 400 C. Then the separated discrete agglomerates are activated to form activated carbon preferably by heating to greater than 800 C. in the presence of steam, carbon dioxide or flue gas.

U.S. Patent No. (Sinclair) evidences the source of an use of expanded carbon particles to prevent prolonged attainment of high concentrations of H₂S in a gaseous environment by a device which comprises a base, an enclosing upper surface which is transparent and water impenetrable, and a light stable composition enclosed by the base and upper surface, said composition undergoing a visible change in color or tone The solutions/suspensions can vary with concentrations and particle sizes from black to purple to amber.

Oxygen turns Vitamin C Yellow then brown then black when oxidized by oxygens.

This oxidation can be mediated by:

1. Pumping from a collapsible bag,

2. Lowering the PH. (L'Oréal patent 7179841B2)

3. Addition of Carbon. (“The use of activated charcoal for the removal of oxygen from gas systems”, R. C. Cooke, Limnology and Oceanography, Vol 18, No. 1, January 1974, Wiley.

Alternative and more effective treatments for the effects of pool chemicals and especially chlorine chemistry are needed. Each of these inventions have fatal flaws not allowing them to be truly effective. Thiosulfates and other formulas react too slowly, Ascorbic acid is not shelf-stabile, oxidizes and turns brown and loses effectiveness.

### SUMMARY OF THE INVENTION

Aqueous solutions, suspensions and dispersions comprising water and a specific class of stabilized ascorbates, especially oil-soluble ascorbates such as bisascorbates such as bis-alkyl ascorbates (e.g., tetrahexyldecyl Ascorbate) and other substituted bis ascorbate compounds (and particularly bisglyceryl ascorbate) and tetraalkyl ascorbates (and with substituted alkyl groups) and bis-aryl ascorbates and other aryl ascorbate. A method of dichlorinating skin, especially dechlorinating skin, hair and/or nails of humans comprising contacting said skin, hair and/or nails with an aqueous composition for sufficient time to reduce halogen effects on the skin, hair and/or nails. The aqueous composition includes at least water and an oil-soluble bis-ascorbate in a weight ratio of about 0.1 to 25% by weight of the total aqueous composition. The composition may be free of ascorbic acid, and the oil-soluble ascorbate is a bis or tri- or tetraalkanol ascorbate such as bisglyceryl ascorbate. There are a variety of delivery methods for this stabile solution depending upon the users situation.

### DETAILED DESCRIPTION OF THE INVENTION

Aqueous solutions comprising a specific generic class of stabilized ascorbates are used in a wide range of care products for humans and mammals to mediate effects from halogen contaminants on skin, especially chlorine deposits and reactants on humans from exposure to chlorinated water. The generic class of ascorbates especially includes oil-soluble ascorbates, such as bisascorbates such as bis-alkyl ascorbates (e.g., tetrahexyldecyl Ascorbate) and other substituted bisacompounds (and particularly bisglyceryl ascorbate) and tetraalkyl ascorbates (and with substituted alkyl groups) with a total of at least 10 carbon atoms per alkyl substituent and from 2 up to 14 carbon atoms per alkyl) and bis-aryl ascorbates and other aryl ascorbates with at least a total of 10 combined total carbon atoms in all of the aryl groups.

The structural formula of bisglyceryl ascorbate is (as a salt, such as a sodium salt thereof), which salts are included within the definition of ascorbates: This is the structure of 3-Glyceryl Ascorbate, and all glyceryl ascorbates are included in the practice of the present invention.

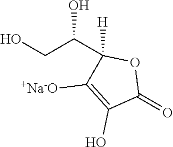

The liquid or gel compositions may be carried on a sheet or film of material, such as natural or synthetic carriers. Cloth, wool, cellulosic sheets, porous sheets, superabsorbent polymer sheets (such as those disclosed in U.S. Pat. Nos. 10,182,946 and 9,901,128 (Gray) and blends of materials can be used to carry the aqueous solutions. “Wet-wipes” with a stabile anti-oxidant is one embodiment for the application of this invention. These can then be used to wipe on the skin to remove chlorine chemicals. In the case of the superabsorbent materials, these can often be reactivated numerous times by application of moisture to partially exhausted sheets and used multiple times, although the solutions will eventually become completely leached from the carrier. These compositions are disclosed in a parent U.S. patent application Ser. No. 16/720,938, filed, 19 Dec. 2020, which is incorporated herein by reference, as are all documents cited in this text. The present technology has found that carbon particles, in the form of coal dust, activated carbon, expanded carbon and other particulate forms of adsorptive or absorptive carbon can be added to the inventor's prior composition and that the suspended attracting (adsorptive or absorptive) particles improved various aspects of the performance of the liquid or gel compositions. The carbon particle size should be, with respect to the geometric mean particle size of the fine particles (especially carbon particles) dispersed in the suspension liquid is less than 0.025 mm, less than 0.02 mm, less than 0.015 mm, and less than 150 microns. The benefits also come with the surprising result that with from 0.05% to 8% by total weight of the composition that as the suspended carbon particles improves chlorine absorption and can make observed removal of the liquid or gel from body or hair surfaces at conclusion of the rinse. The ambifunctional (both oleophilic and hydrophilic) structure in the modified Vitamin C acts to help stabilize the carbon particles, especially the activated and expanded carbon (which tends to have ionic components in them) and keep them in suspension.

The ascorbates may have Na, Li, Ca, K, NH₄ and other cations as the salt alternatives. The simplest solution useful in the practice of the present technology would be water and the water-dispersible to water-soluble ascorbates described in the practice of the present invention. Additives typical for use in shampoos, skin cleansers, skin washes, hand washes and soaks can then be added to the basic formulation to make specific classes of products. Shampoo, Soaps, Body Wash, Lotions, Gels, and Sprays.

The final compositions will contain from about 0.1 to 35% by total weight of the composition as the oil-soluble ascorbate with the remainder of the 100% of the compositions comprising water and typical care additives that would not adversely react with the oil-soluble ascorbates. Such typical ingredients include surfactants, thickening agents, fragrances, moisturizers, anti-inflammatories, dyes, pigments, UV-absorbers, and the like.

A dark colored (Black or Deep Blue) body wash with Anti-Oxidant properties can be prepared with the attractive carbon particles for the removal of chlorine affixed to human skin and hair. (Free Hypochlorous Acid and combined chlorines from a pool), bromine and other halogens. Using activated charcoal helps remove oxygen as well as make the solution darker in color and reduce oxidation of components in the liquid or gel. Although Chlorine and Bromine are useful chemicals, sanitizing and oxidizing contaminants in a pools, drinking water, municipal water and hot tubs, when swimmers enter the pool it affixes to the skin and hair, causing itching, discomfort and an displeasing smell. It can also lead to disfiguring skin disease such as chloracne. Normal shampoos do nothing to remove this powerful Halogen+skin/hair bond. Regular use can cause hair to turn stiff, damaged and discolored. Solution. Vitamin C is a powerful anti-oxidant that oxidizes when in contact with Oxygen in the atmosphere. One study and subsequent patent shows a desirable minimum 3.7% solution is needed to be applied to the skin to allow the bonds to be broken. Using a 10% solution can counteract the loss of effectivity of Vitamin C due to oxidation. Carbon including Powdered Activated carbon also can provide a mild abrasive to get into the pours. A deep blue or black coloring can obscure the browning and pH can be balanced to impede irritation to the eyes. Browning of vitamin C by control of the pH (keeping it slightly acidic) can be reduced and optimized to reduce oxidation.

Even though controlled air flow back into the liquid or gel container (e.g., a valve) can be used to reduce the amount of oxygen by reducing the amount of oxygen in the container. A bag with a voiding pump can be used to reduce the amount of Oxygen Activated Carbon is a natural oxygen absorber. Other Oxygen absorbing chemicals (Such as activated carbon) can be added to remove the free oxygen in the canister. Other chemicals such as oxy-sorb, an Oxygen absorber that looks like sodium also helps in their solution Vitamin C comes in many forms and can be mixed in a sundry of ways. 1. Ascorbic acid is the most commonly consumed and also the least expensive form of vitamin C available. However, its slight acidic component can make it hard on the digestive system for some, especially those with stomach acid issues. Many studies use this formulation of vitamin C. While ascorbic acid is synthetically made, it is identical to the formulations found in nature. Since studies show only 30 percent of a given dose is actually absorbed, researchers have also sought other formulations that may be better absorbed in the gastrointestinal tract. Ascorbic acid is available in tablets, capsules, or as a powder. There are also many mineral ascorbates. • Calcium Ascorbate that can be used as supplemental additives. This formulation contains both calcium (at 100 mg) and ascorbate (at 900 mg) • Magnesium Ascorbate—This formulation contains both magnesium (50 to 100 mg) and 900 mg of ascorbate. • Sodium Ascorbate—This formulation contains both sodium (˜100 mg) and 900 mg of ascorbate. 2. Ascorbate 3. Vitamin C Metabolites (Ester-C®) is a patented formulation of calcium-ascorbate that was discovered in the 1980s. It contains a small amount of vitamin C metabolites such as calcium threonate, xylonate, and lyxonate, as well as dehydroascorbic acid. 4. Liposomal Vitamin C is a formulation that appears to have improved bioavailability or absorption properties. To help increase absorption, scientists have developed liposomal vitamin C 5. Ascorbyl Palmitate: This formulation allows vitamin C, which is normally water-soluble, to become fat-soluble. It is usually added to topical vitamin C preparations so that it can be absorbed in the skin. 5. Stabilized Vitamin C (as in our previous shampoo and conditioner patent.) as well as many other versions. 6. Mixing of Ascobic acid can also stabilize it. Ascorbate and other derivatives. Additional chemicals, such as preservatives, surfactants, vitamins, anti-inflammatants (hydrocortisone) ingredients for acne.

General detergent anionic-cationic surfactant mixtures are well known to the art. See generally, U.S. Pat. Nos. 5,441,541, 5,472,455, 5,204,010, 4,790,856, 4,298,480, 3,730,912 (all to The Colgate-Palmolive Company), U.S. Pat. Nos. 5,622,925, 5,607,980, 5,565,145, 4,913,828, 4,659,802, 4,436,653, 4,338,204, 4,333,862, 4,132,680 (all to The Procter & Gamble Co.); also see WO 97/03164, WO 97/12022 and WO 96/37591 (all to The Procter & Gamble Co.), and WO 97/28238 and WO 97/15647 (both to Reckitt & Colman, Inc.). See also, U.S. Pat. Nos. 5,610,187 and 4,247,538 (both to Witco Corp.), U.S. Pat. No. 5,344,949 (to Th. Goldschmidt AG), U.S. Pat. Nos. 5,332,854 and 5,324,862 (both to Dai-Ichi Kogoyo Seiyaku Co., Ltd.), U.S. Pat. No. 4,273,760 (to National Starch and Chemical), and U.S. Pat. No. 4,264,457 (to DeSoto, Inc.).

Compositions comprising anionic-cationic surfactant mixtures are also relatively well known. U.S. Pat. No. 6,007,802 discloses a conditioning shampoo composition with excellent cleaning performance and improved levels of conditioning while minimizing any adverse effect associated with build-up; the disclosed compositions generally comprise an ethoxylated alkyl sulfate, amphoteric surfactant, insoluble, dispersed conditioning agent (nonionic, cationic silicone), synthetic esters, and cellulosic cationic polymers. U.S. Pat. No. 5,939,059 discloses a 2-in-1 conditioning shampoo comprising an anionic surfactant (alkyl sulfate or ether sulfate) and ester quats, with optional amide. U.S. Pat. No. 5,747,436 discloses a low static conditioning shampoo comprising an anionic and an amphoteric surfactant, complex acid:amine (1:1 mole ratio) and polyquaternary compound. U.S. Pat. No. 5,607,980 discloses topical compositions having improved skin feel comprising an anionic surfactant (alkyl sulfate, ether sulfate, isethionate), a cationic surfactant and an amphoteric surfactant. U.S. Pat. No. 5,997,854 discloses a conditioning shampoo formulation comprising a quaternary ammonium component, an emulsifier, an amphoteric, an alkyl polyglycoside surfactant. U.S. Pat. No. 5,145,607 discloses an optically clear conditioning shampoo comprising anionic (alkyl sulfate or alkyl ether sulfate) and cationic surfactants. U.S. Pat. No. 4,931,216 discloses detergent compositions comprising an anionic or amphoteric surface-active agents and a branched quaternary ammonium salt. Cationic surfactants may also be used. U.S. Pat. No. 4,744,977 discloses quaternary ammonium compound hair conditioners in combination with an anionic surfactant. U.S. Pat. No. 5,661,189 discloses mixtures of anionic, cationic, amphoteric, nonionic, zwitterionic surfactants, along with benefit agents, thickening agents in small amounts of soap. These references are incorporated herein by reference for their general disclosure and compounding methods.

While ascorbic acid is very beneficial to the skin, it is notoriously difficult to stabilize in cosmetic formulations. This is because it is such an excellent antioxidant, which means that it will readily donate electrons to the oxygen content in air. After becoming oxidized, the ascorbic acid molecule is ineffective and cannot provide skin benefits. Therefore, a product that contains ascorbic acid needs to be packaged to prevent oxidation. When purchasing a product containing ascorbic acid, make sure that the packaging is opaque and is either an airless pump bottle or a tube. (Zero contact with oxygen, inert gases such as Nitrogen)

The safety of ascorbic acid and its salts has been assessed by the Cosmetic Ingredient Review (CIR) Expert Panel. The Panel reviewed the scientific data and concluded that these ingredients were safe for use in cosmetic and personal care products. Since ascorbic acid is an acidic ingredient, some people may experience sensitivity, redness, or stinging after using products with this ingredient. These side effects typically occur when high concentrations of ascorbic acid are used, or when ascorbic acid is combined with other ingredients that exfoliate the skin. The oil-soluble ascorbates of the present invention are also more gentle on the skin and hair than ascorbic acid.

Furthermore, chlorine (including, for example, gaseous or solvated Cl₂, chlorine-comprising oxidizing agents, and salts thereof) has a multitude of uses in both household and industrial applications. For example, it can be used for disinfecting, whitening, bleaching, and clarifying materials. Chlorine is often used as an antimicrobial agent. For example, sodium hypochlorite (a chlorinating agent) is known to kill a broad array of microbes. Owing to the efficacy, cost, and versatility of chlorinating agents, they are amazingly attractive reagents for a variety of home and industrial applications. These non-pool applications can also cause similar effects to pool chlorine when the materials are spilling on persons or merely adsorbed from the air.

A down side to using chlorinating agents in the home and industry is that they can react with many of the materials to which they are exposed—often materials that the user would like to keep free from chlorination. Because materials and surfaces that are exposed to chlorine and chlorinating agents undergo a chemical reaction with the chlorine, their chemical composition becomes altered. Part of the chlorine and/or chlorinating agent becomes bound to the material or surface. Accordingly, one cannot simply wash away the residual chlorine. The bound chlorine must first be liberated before it can be washed away with traditional soaps and surfactants. The present compositions, even though designed for use on skin, hair and/or nails, may also be used to wash surfaces in an industrial setting where chlorine compounds may deposit.

In the food industry, it may be desired to chlorinate the water that is used to chill poultry carcasses, such as chicken, but chicken producers would like to avoid chlorinating the biological tissues making up the chicken. Poultry is an important part of the worldwide animal food market. The poultry industry raises chickens, kills them, and then processes them into a form that is both convenient and safe for the consumer to use in preparing meals. Converting live chickens into healthy food presents challenges to the chicken industry. In particular, poultry provides an excellent medium for the growth of microorganisms, such as Pseudomonas, Staphylococcus, Micrococcus, Acinetobacter, Moraxella, and Salmonella. Workers can easily have their hair and nails absorb these chlorine compounds of have their skin adsorbed with the compounds.

In various embodiments, salts and/or derivatives (including but not limited to oxidized forms) of ascorbates may be used in place of, or in addition to the preferred bisglycedyl ascorbate. Salts and/or derivatives of these oil-soluble compounds would function similarly within the context of the disclosed compositions and methods. As non-limiting examples, esters or ethers of the ascorbate may be used in the compositions and methods of the disclosure. Unless otherwise noted or individually recited, when the term “ascorbic acid” is used herein, it is intended to include salts (e.g. sodium, potassium, calcium, etc. of the ascorbate) and/or degradation products and/or derivatives (e.g., oxidized forms) thereof, whether or not so stated. For example, when present within an aqueous solution, the term “ascorbate” as used herein is intended to include products formed as a result of ascorbate reacting with oxygen dissolved in water and/or oxygen.

Furthermore, as known, ascorbic acid has stereogenic centers in its structure, for example the carbons modified by the (R)- and (S)-indicators in the chemical name (R)-3,4-dihydroxy-5-((S)-1,2-dihydroxyethyl)furan-2(5H)-one. This disclosure contemplates use of any combination of stereoisomers of ascorbic acid as the base of the ascorbate, including isolated stereoisomers, and all mixtures thereof.

The phrases “effective amount of ascorbic acid” or “effective amount of a composition comprising ascorbic acid”, and variations thereof, as used herein, are intended to include any amount of ascorbate (as defined herein to include salts, degradation products, and derivatives) that is sufficient to mediate or even convert any or all of the chlorine compounds (chlorine, chloride, hypochlorite, etc.), including N—Cl bonds or groups adsorbed or bound to the hair, skin or nails being treated into N—H. In at least one embodiment, an effective amount of ascorbic acid means an amount sufficient to mitigate (i.e., reduce to any degree or eliminate completely) the perceptible adverse effects of exposure to the chlorine compounds. For example, in one embodiment, an effective amount of ascorbic acid would be an amount sufficient to reduce or eliminate an odor of chlorine from hair, skin and/nails, such as, for example, to reduce or eliminate an undesirable “pool odor” present on the body and/or clothes of a swimmer. In compositional amounts, the liquid aqueous compositions may comprise from 0.05% by weight up to 30% or 0.10% to 25%, 1.0% to 25%, 2.0% to 20 percent, or 2 t0 20% by weight of the oil-soluble ascorbate to 100 parts by weight of total aqueous liquid or gel composition. In using gels, where the material is a hair wash, foaming agents may be added to increase the visual foaming effects users expect. Alternatively, the liquid aqueous compositions may comprise from 0.5% by weight up to 20% by weight of the oil-soluble ascorbate to 100 parts by weight of total aqueous composition or liquid aqueous compositions may comprise from 1.0% by weight up to 18% by weight of the oil-soluble ascorbate to 100 parts by weight of total aqueous composition. In a further functional exemplary embodiment, an effective amount of ascorbic acid is an amount sufficient to reduce the number of N—Cl bonds in hair, on skin or in nails by at least about 50%, 60% or even 90% on a molecular number basis.

In further exemplary embodiments, a solution according to the disclosure may comprise a concentration of ascorbic acid ranging from about 0.5 to about 2 Molar. By way of non-limiting example, aqueous solutions may comprise about 0.3 to about 3 moles, such as about 0.5 to about 1.5 moles, or about 1.5 to about 3 moles, of compounds of formula A per one liter of water. Concentrates to be later mixed with water to compositions of these active levels of ascorbates may even have higher concentrations.

In exemplary embodiments where treatment compositions are applied to reduce or eliminate adverse effects of exposure to chlorinating and/or brominating agents, e.g., the odor of chlorine and/or itching and/or skin irritation, the effective amount of ascorbate may vary. Choosing an effective amount is within the skill of those of skill in the art, and may, for example, be done empirically, using more ascorbic acid where the odor of chlorine and/or itching/and/or skin irritation persists after applying an initial amount of ascorbate. By way of example only, an effective amount of ascorbate may range from about 0.2 to about 10 grams, such as about 0.5 to about 5 grams, or such as about 1 to about 3 grams per 0.1 liters of water. In certain of the various exemplary embodiments, the compositions and methods disclosed herein comprise ascorbic acid solutions having a pH of from 6.0 to 7.5, although in treatment of fabric or surfaces, the pH may be less than about 6, such as, for example, less than about 5, less than about 4, or less than about 3. In further embodiments, the ascorbic acid solutions may have a pH of between about 3 and about 4. In yet further exemplary embodiments, the ascorbic acid solutions may have a pH of about 2. In one embodiment, the pH of the ascorbic acid solutions may range from about 1.9 to about 2.2. In further exemplary embodiments, the compositions and methods disclosed herein comprise ascorbate solutions, dispersions, suspensions or emulsions having a pH in the range of about 5 to about 8, such as about 5 to about 7.5, about 6 to about 7, about 7 to about 7.5, or about 6. Compositions having a particular pH and concentration of ascorbic acid may be made by applying the Henderson-Hasselbalch equation for estimating the pH of a buffer solution. Baby Shampoos, lotions and conditioners need to be pH balanced with addition of the buffers, salts, acids or bases.

In yet further exemplary embodiments, the ascorbate used in the compositions and/or methods described herein may be replaced with, or used in combination with, one or more acid, salt, and/or derivatives thereof, to form acidic solutions, such as, for example, acidic aqueous solutions. Any acid, salt, and/or derivative thereof that is safe for the intended application may be used. For example, one or more of the following acids may be used in an acidic aqueous solution contemplated by the disclosure: acetic acid, citric acid, aconitic acid, adipic acid, benzoic acid, caprylic acid, cholic acid, desoxycholic acid, erythorbic acid, ferulic acid, formic acid, glutamic acid, glycocholic acid, hydrochloric acid, lactic acid, linoleic acid, malic acid, nicotinic acid, oleic acid, pectinic acid, phosphoric acid, propionic acid, sorbic acid, stearic acid, succinic acid, sulfamic acid, sulfuric acid, tannic acid, tartaric acid, taurocholic acid, and/or thiodipropionic acid. As a further example, one or more of the following salts may be used in the compositions according to the disclosure: lauryl sulfate salts, laureth sulfate salts, and sodium ascorbate. The compositions according to further embodiments may also comprise glycerin and/or menthol. As such, throughout the disclosure where compositions and methods comprising ascorbic acid, salts, and/or derivatives thereof are described, it should be understood that the ascorbate, salts, and/or derivatives may be replaced with, or used in combination with, one or more acid, salt, and/or derivative thereof.

Exemplary compositions according to the disclosure may be aqueous, and may be in any known form. For example, they may be solutions, powders (to later form aqueous compositions), tablets (to later form aqueous compositions), creams, gels, emulsions, etc. The compositions according to various embodiments of the disclosure may be packaged in any type of vessel or container that is useful for the specific formulation, and effective to dispense the composition. In at least certain exemplary embodiments, the vessel containing the composition according to the disclosure may be free or substantially free of O₂ for stability. A Bag on Valve, application. For example, the composition may be applied or administered by dispensing or spraying it from an aerosol or non-aerosol container that is free or substantially free of O₂.

In one embodiment, for example, the compositions may be in the form of a tablet, such as an effervescent tablet, which may be placed in water or another solvent before use. In yet a further exemplary embodiment, the composition may be an aqueous solution. There are a number of different commercial embodiments that are considered in the practice of the present technology, including at least:
1. Lotion/conditioner using Bisgeryl-C
2. shampoo/body wash using Bisgeryl-C
These are the current products and neutralise free chlorine in solution
3. Black or Blue Body wash using a ˜5% vitamin C (ascorbic or ascorbate), additional carbon to remove oxygen from solution. PH of 3 to 7.4 in Oxygen reducing packaging.
4. Vitamin C gel (like Aloe) for after sun or after pool using enough Vitamin C to neutralise chlorine 3.7%
6. Vitamin C and Carbon bar of soap.

Exemplary soap compositions according to the disclosure may be made, for example, by mixing (A) an aqueous solution of ascorbate as described herein with (B) a liquid soap composition. In various embodiments, the soap compositions according to the disclosure have a pH of less than about 6, such as, for example, less than about 5, less than about 4, or less than about 3. In further exemplary embodiments, the soap compositions according to the disclosure have a pH in the range of about 5 to about 8, such as about 5 to about 7.5, about 6 to about 7, about 7 to about 7.5, or about 6. In another exemplary embodiment, the composition may be a foaming composition. Foaming compositions contemplated herein may comprise, inter alia (what is inter alia?), water, ascorbate, and a foaming agent.

Exemplary foaming compositions according to the disclosure may be made, for example, by mixing an aqueous solution of ascorbate with a foaming agent (e.g an appropriate shampoo, soap, or body wash) to form a liquid that is capable of providing a foam when dispensed through a foaming hand soap dispenser. In various embodiments, the foaming compositions according to the disclosure have a pH of less than about 6, such as, for example, less than about 5, less than about 4, or less than about 3. In further exemplary embodiments, the foaming compositions according to the disclosure have a pH in the range of about 5 to about 8, such as about 5 to about 7.5, about 6 to about 7, about 7 to about 7.5, or about 6.

In addition to ascorbate, other component(s) useful in compositions according to the intended application, as long as the additional component(s) do not substantially interfere with the intended function of the ascorbic acid. By way of non-limiting example, for applications intended to be applied to body and/or clothing fibers, such additional components may include, but are not limited to, emollients, preservatives, perfumes, thickeners, cleansing agents, etc. It may also be desirable in various embodiments that the additional components do not damage or otherwise adversely affect the skin, hair and/or nails to which the composition is applied. Additional ingredients beneficial to skin, Vitamins A,D,B and E as well as Hylauranic acid, moisturizing oils, essential oils can be added to the invention in solutions involving leave on scenarios. Because all Municipal water in showers and baths have chlorine as a disinfectant in water towers, use of the invention post shower/bath is necessary.

One of skill in the art would be able, through routine experimentation using the information provided herein, to formulate acceptable compositions comprising an effective amount of ascorbate, salts, and/or derivatives thereof, and formulations thereof, for treatment of chlorinated or chlorine-affected surfaces. As non-limiting examples, formulations comprising ascorbate in a molar ratio of about 79:1, or comprising water:ascorbate acid in a molar ratio of about 1:55.6.

Further aspects of the disclosure provide methods of mitigating adverse effects of exposure to oxidizers such as chlorinating and/or brominating agents, e.g. swimming pool chemicals and associated itching, irritation, and/or “pool odor.” The composition according to the disclosure may be sprayed by any method known, such as, for example, an aerosol spray, pour bottles, squeeze bottles or a non-aerosol pump bottle. In at least one exemplary embodiment, the composition according to the disclosure is applied by using a foaming composition comprising ascorbic acid, water, and a foaming agent. In some exemplary embodiments, the foaming composition is applied by dispensing said foaming composition through a dispenser (e.g. a foaming hand- or body-soap, or shampoo, dispenser), thereby creating a foaming lather, which is applied to the hair and/or skin. “No touch” dispensers can be used to administer a gel, liquid or foam of the invention.

In at least one exemplary embodiment of a subject exposed to chlorine and/or bromine in a swimming pool, the subject may optionally first rinse the residual pool water from his/her hair and/or body, then apply a composition according to the disclosure to his/her hair and/or body. In some embodiments, the person may thereafter immediately or substantially immediately wash the hair and/or body with soap, or may apply shampoo and/or conditioner. In further embodiments, the composition according to the disclosure is sprayed onto the hair and/or body after rinsing off residual pool water without subsequently immediately or substantially immediately washing the hair and/or body. This disclosure also contemplates applying a composition according to the disclosure without first rinsing. When a composition according to the disclosure is applied without first rinsing, larger quantities may, in at least some embodiments, be required than when applied subsequent to rinsing.

In at least one exemplary embodiment, the method of applying a composition according to the disclosure is intended to include a method where a subject, such as a swimmer, applies an amount sufficient to reduce and/or eliminate his/her own “pool odor” and/or irritation as perceived by the subject. For a particular aqueous solution according to the disclosure, the effective amount required to reduce and/or eliminate the subject's odor and/or irritation may depend on the amount of hair and/or skin that the subject has. Generally speaking, a subject may adjust the amount of the composition applied based on his or her own observations—if the subject experiences “pool odor” and/or irritation following administration, more may be applied. In various embodiments, the methods according to the disclosure relate to methods of treating hair comprising applying a composition as described herein. It is contemplated that a person with little or no hair would recognize that using a smaller amount of the composition provides effective reduction or elimination of “pool odor” and/or irritation, and thus, in at least certain embodiments, an effective amount may be less than in an embodiment where a person has a lot of hair and skin. (The use of dechlorination Glyceryl Ascorbates in post-shower, after bath, after pool/hottubs is necessary because 99% of all showers using municipal water contains chlorinated water.) After shower sprays, conditioners and lotions are helpful at lower levels. Likewise exposure to higher concentrations of halogens require stronger concentrations of the invention, double strength, triple strength etc.

The compositions according to the disclosure may, in various embodiments, be applied to the body and/or clothing fibers and immediately removed (e.g. by rinsing the body right away, that is in less than one minute), or may be left on the fibers for a period of time after application. For example, the body may be washed (e.g. with soap or shampoo) subsequent to the application of the composition.

As described herein, the disclosure is set forth with regard to “people” for ease of reference only. However, the invention is not limited to humans, but rather is intended to include any mammal having hair. As such, the use of the terms “swimmer” or “people” is intended to include any mammal that swims or is otherwise exposed to swimming pool chemicals such as chlorine and/or bromine.

The terms “pool chemicals” and the like are used generally throughout the disclosure for ease of reference, but should not be considered limited to chemical exposure in a swimming pool. Rather, any chemical such as chlorinating agents (e.g., hypochlorite salts such as calcium hypochlorite or sodium hypochlorite, hypochlorous acid) or brominating agents (e.g., bromine, salts comprising bromine, hypobromous acid, etc.) or other oxidative halogens are intended to be included with reference to “pool chemicals” and the like, whether or not a person is actually exposed to those chemicals in a swimming pool.

The term “biological fibers” is intended to refer to fibers comprising proteins, such as those found in and from living beings, including but not limited to skin, hair, nails, muscle, and other fibrous tissues of mammals, as well as plant fibers. It is also intended that fibers from harvested animals that are no longer living, such as harvested poultry during processing, are included in the term “biological fibers,” as these fibers likewise comprise proteins.

Further embodiments of the disclosure are related to methods for treating biological and/or synthetic fibers, such as may be found in some textiles, e.g. towels, swimming attire, swimming accessories, etc., exposed to oxidizing agents, such as those exposed to chlorinated and/or brominated pool water, with an aqueous solution of ascorbic acid. Such treatment may reduce the “pool odor” of, and/or “wear” associated with such exposure on, the textiles. For example, treating swimming attire exposed to chlorinated and/or brominated pool water with a composition according to the disclosure has been found to reduce the “pool odor” of that swimming attire. Treating swimming attire exposed to chlorinated and/or brominated pool water with composition according to the disclosure has also been found to reduce the oxidative damage done to the swimming attire, thereby prolonging its life and reducing the fading and/or discoloration of its materials, typically associated with such textiles. As used herein, the terms “clothing” and “textiles” should be considered interchangeable in that when the term “clothing” is used, it is intended to include any textiles within the scope of the disclosure. For example, when reference is made to treatment of a swimmer's clothing or attire, it is intended that a swimmer's towel, etc., is included in said disclosure and claims. It should also be noted that textiles are intended to be included as biological and/or synthetic fibers, as textiles are traditionally made up of fibers such as plant and/or animal fibers, or may be manmade, such as polymer fibers.

In at least one exemplary method, an effective amount of a composition according to the disclosure is applied by treating the textiles with an aqueous solution of ascorbate, such as by applying the composition by contacting the textiles with said composition. Any method for treating textiles known in the art may be used, such as, for example, spraying the textile with the composition using an aerosol or non-aerosol spray; immersing the textile into the composition; and/or washing the textile with a soap composition comprising ascorbic acid, as described herein. In one exemplary embodiment, the textiles may optionally first be rinsed with standard tap water before applying the composition according to the disclosure, and optionally thereafter be rinsed a second time with standard tap water after the application. In another embodiment, the composition according to the disclosure is applied without first rinsing the textiles with standard tap water. The textiles may optionally be washed as normal, subsequent to the application of the composition according to the disclosure. In at least one exemplary embodiment, the textiles may be treated with about 0.2 to about 10 grams of ascorbate carried in water, such as about 0.5 to about 5 grams of ascorbate, or about 1 to about 3 grams of ascorbate.

In further embodiments according to the disclosure, methods for reducing biological fibers are also contemplated. Such methods include steps of (1) identifying biological fibers which are capable of undergoing reduction, and (2) applying a composition according to the disclosure to said biological fibers. In further embodiments, said methods include a step of applying a composition according to the disclosure to said biological fibers.

In further embodiments according to the disclosure, methods of administering vitamin C to a mammal are contemplated, said method comprising administering a composition according to the disclosure via a vessel that is free or substantially free of O₂. Ascorbic acid is known to degrade when allowed to stand in the presence of oxygen. Oxygen is a natural component of the Earth's atmosphere, where it resides in air and water. It has been found that storing the compositions according to the disclosure in an oxygen-free environment provides for many advantages, such as prolonging storage and shelf life.

Aqueous ascorbate gels, emulsions or solutions according to various embodiments of the disclosure may thus optionally be made oxygen-free, for example by bubbling an inert gas through the solution, thereby driving out any oxygen dissolved in the water. Alternatively, aqueous ascorbate solutions may be made by mixing the ascorbate with water, sealing the solution off from the environment (e.g., by sealing the solution in a bottle), and allowing the bottle to stand until the oxygen sealed inside the bottle is consumed by the ascorbate in the solution. By way of example only, methods of administering ascorbate to mammals may comprise dispensing a composition according to the disclosure by spraying said composition, e.g. using an aerosol spray or a non-aerosol pump, foaming chamber, (Aloe lotions with glyceryl Ascorbate(s)), Shaving creme, Suntan lotion, and After-Sun lotions.

Maintaining free chlorine in recommended levels is the most important activity in terms of ensuring your pool and hot tub are clean and safe to use. However, recommended levels are not necessarily the same for both pools and hot hubs. Further, recommended levels are dependent on cyanuric acid, temperature and bather load. And the effectiveness of free chlorine's ability to kill contaminants is greatly dictated by the pH level of the water. The following discussion takes some of these factors into consideration to determine chlorine levels that are typically in pools to assist in determining the efficiency of the compositions of the present invention.

### Ideal Levels of Free Chlorine, Combined Chlorine, and Total Chlorine

The Association of Pool and Spa Professionals recommends free chlorine levels be kept between 2.0 and 4.0 ppm. The Center for Disease Control recommends free chlorine stay above 1 ppm in pools and 3 ppm in hot tubs. Municipal water towers are required to keep chlorine above 0.5 ppm to guard against legionella and other pathogens, most cities maintain between 1 and 2 ppm.

The easiest way to check chlorine levels is with test strips. Free chlorine is the amount of chlorine freely available to kill bacteria and other contaminates. Chlorine bound by contaminates are called Combined Chlorines, some of these can be very toxic.

The recommended amount of “combined chlorine” is no more than 0.2 ppm. Combined chlorine or chloramines is the portion of chlorine that has reacted with the water and microorganisms contained in it. It is a temporary form of chlorine that should be oxidized if there is at least than 7.5 ppm of free chlorine in the water to burn up the contaminates. Too much combined chlorine (i.e. more than 0.5 ppm) means that there is not enough free chlorine available to kill all the contaminates and causes severe skin, lung and eye irritation as it is emitted from the pool. When combined chlorine reaches these levels, the water may cause skin and eye irritation, be harmful to swim in due to bacteria, and may allow algae to form. The present technology in sufficient concentration will also attack and destroy the more complex N-CL bonds of Combined Chlorine.

The present technology has a rapid and significant effect on the chlorine products in hair (which is the easiest to determine standards because of greater absorption consistency), and these tests may also be used with skin products. For example, human hair has an average thickness of from 17 to 181 micrometers (millionths of a meter). A basic test would be for human hair having an average thickness of from 50-100 micrometers to be immersed in chlorinated water having 2 ppm free chlorine and less than 0.5 ppm combined chlorine for 15 minutes. Tins hair (whether removed from a human scalp and retained in a clip at one end or still on the head of a human) is then treated (washed or soaked) for 1.5-2.0 minutes with the hair (or skin) treating composition of the invention and rinsed thoroughly in tap water with scrubbing to remove at least 95% by weight of the applied composition. The hair is then leached in 1.0 liters of deionized water for 2 minutes and the free chlorine level and the combined chlorine level measured. The free chlorine level must be below 0.1 ppm and the combined chlorine level must be below 0.05 ppm to pass this test. An additional test can be to administer the present technology to a solution of chlorinated/brominated water, with concentration of greater 2 ppm and less than 0.5 ppm oi combined chlorine. Effectiveness can be measured by the time requited for the anti-oxidation of the halogens in the water. This test can be compared and contrasted to the extant products on the market.

Although the present disclosure herein has been described with reference to various exemplary embodiments, it is to be understood that these embodiments are merely illustrative of the principles and applications of the present disclosure. Those having skill in the art would recognize that various modifications to the exemplary embodiments may be made, without departing from the scope of the invention. Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

It will be appreciated that there is an implied “about” prior to all numerical values recited herein, whether or not so stated. It should also be understood that the precise numerical values used in the specification and claims form additional embodiments. Efforts have been made to ensure the accuracy of the numerical values disclosed herein. Any measured numerical value, however, can inherently contain certain errors resulting from the standard deviation found in its associated measuring technique. As used herein, reference to percent is intended to indicate wt %, relative to the weight of the composition.

Furthermore, other embodiments of the disclosure will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. Thus, by way of example only, reference to “a composition” can refer to one or more compositions, and reference to “a salt of ascorbate” can refer to one or more salts of ascorbate. As used herein, the terms “comprise”, “comprises”, “comprising”, “contain”, “contains”, “containing”, “have”, “having”, “include”, “includes”, and “including” are intended to be non-limiting, such that recitation of an item or items is not to the exclusion of other like items that can be substituted or added to the recited item(s).

### EXAMPLES

The following examples are illustrative only, and are not intended to be limiting of the invention, as claimed.

### Example 1

At standard temperature and pressure, 330 grams of bisglyceryl ascorbate or its sodium salt were dissolved in 1 L of water. This solution 1 was then applied to human hair and human skin that had been exposed to swimming pool chemicals, oxidizers or halogens. An equivalent solution can be made by low shear blending of crushed (0.05 mm) expanded carbon to form a suspension of the carbon in the liquid to form a more stable product.

### Example 2

At standard temperature and pressure, 300 grams of bisglyceryl ascorbate were dissolved in 1 L of water along with coco betaine. This solution 2 was then applied to hair and human skin that had been exposed to swimming pool chemicals. An improved solution/suspension can be formed by adding small particles (0.025 mm) of activated carbon with low shear stirring (to avoid foaming) to form a stable, more oxygen resistant skin or hair wash composition.

### Example 3

At standard temperature and pressure, 270 grams of ascorbate were dissolved in 1 L of water along with citric acid. This solution 3 was then applied to human skin and human hair that had been exposed to swimming pool chemicals. An improved solution/suspension can be formed by adding small particles (<0.005 mm) activated carbon or coke dust with low shear stirring (to avoid foaming) to form a stable, more oxygen resistant skin or hair wash composition.

### Example 4

At standard temperature and pressure, 240 grams of ascorbic acid were dissolved in 1 L of water along with an emulsifying agent and lemon oil. This solution 4 was further modified by adding small particles (<0.005 mm) activated carbon or coke dust with low shear stirring (to avoid foaming) to form a stable, more oxygen resistant skin or hair wash composition. This was stored in a hand spray pump bottle for three days and then was then applied to human skin and human hair that had been exposed to swimming pool chemicals.

### Example 5

At standard temperature and pressure, 180 grams of ascorbic acid were dissolved in 1 L of water. This solution 4 was further modified by adding small particles (<0.005 mm) activated carbon or expanded carbon with low shear stirring (to avoid foaming) to form a stable, more oxygen resistant skin or hair wash composition. This solution 5 was then applied to human hair and human skin that had been exposed to swimming pool chemicals.

### Example 6

At standard temperature and pressure, 180 grams of ascorbic acid were dissolved in 1 L of water along with an emulsifying agent, coco betaine and peppermint oil. This solution 4 was further modified by adding small particles (<0.005 mm) activated carbon or coke dust with low shear stirring (to avoid foaming) to form a stable, more oxygen resistant skin or hair wash composition. The resulting solution 6 was transferred into 3 oz spray bottles. Following swimming in a chlorinated swimming pool, a subject's skin and hair were rinsed with warm shower water for about 30 seconds. Then, the solution 6 was applied to the subject by spraying the solution onto the skin and hair. A total of 10 mL of solution 6 was evenly sprayed onto the subject's skin, and a total of 20 mL of 6 solution 12 was evenly sprayed onto the subject's hair. The subject was then allowed to wash and rinse their skin with soap, and shampoo, rinse, and condition their hair, as normal.

The shampoo was applied to the hair without first rinsing out the solution 6, so that the shampoo and lather distributed the solution.

Other ingredients may include propylene glycol, sodium benzoate, glycerine, DMDM (3′-Demethoxy-o-Demethylmatairesinol), hydantoin imadazolininyl, sodium benzoiate, 2-bronio-2-nitropropane-1-2-diol. There are a whole range of products dechlorinating in the line of compositions useful in the disclosed invention, such as in shampoo, Hand Soap, Body-Wash, Bubble bath, Baby Shampoo (liquid, gel, foam); shampoo and conditioner; Conditioner, Leave in Conditioner, De-tangler, after shower spritz (liquid conditioner sprays); De-chlorinating Lotion moisturizer, Aloe gel, shaving creme, shaving foam, wet-wipes and the like.

Additions to the formulae include many special materials that are good for skin such as Vitamin A&D, anti-oxidant Vitamins C and E, as well as Hylauranic acid, essential oils etc. These additional ingredients may be used both in Soap and Conditioner and lotion forms.

A maximum effective formula would use a wide range of materials such as the basic formulations of the above examples with using anything else that chemically address chlorine activity or even the chlorine/Bromine bonds, such as thiosulfate salts, chelating agents for chlorine and other ascorbates.

What is claimed is:

1. An aqueous composition comprising water and an oil-soluble bis-ascorbate in a weight ratio of about 0.1 to 25% by weight of the total aqueous composition and 0.05% to 30% by weight of the total aqueous composition of suspended carbon particles.

2. The aqueous solution of claim 1 wherein there is 0.1% to 25% by weight of suspended carbon particles selected from the group consisting of activated carbon and expanded carbon.

3. The composition of claim 1 that is free of ascorbic acid.

4. The composition of claim 2 that is free of ascorbic acid.

5. The composition of claim 2 consisting essentially of water, ascorbic acid, the suspended carbon particles and bisglyceryl ascorbate.

6. The composition of claim 3 consisting essentially of water, ascorbic acid, surfactant and bisglyceryl ascorbate.

7. The composition of claim 2, having a pH of about 6 to about 7.5.

8. A method of dehalogenating skin, hair and/or nails of humans comprising contacting said skin. Hair and/or nails with the aqueous composition of claim 1 for sufficient time to reduce halogen effects on the skin, hair and/or nails.

9. The method of claim 8 wherein the method dechlorinates skin and/or hair.

10. A method of dechlorinating skin, hair and/or nails of humans comprising contacting said skin, hair and/or nails with the aqueous composition of claim 2 for sufficient time to reduce halogen content on the skin, hair and/or nails.

11. A method of dechlorinating skin, hair and/or nails of humans comprising contacting said skin, hair and/or nails with the aqueous composition of claim 3 for sufficient time to reduce halogen effects on the skin, hair and/or nails.

12. A method of dechlorinating skin, hair and/or nails of humans comprising contacting said skin, hair and/or nails with the aqueous composition of claim 4 for sufficient time to reduce halogen effects on the skin, hair and/or nails.

13. A method of dechlorinating skin, hair and/or nails of humans comprising contacting said skin, hair and/or nails with the aqueous composition of claim 5 for sufficient time to reduce halogen effects on the skin, hair and/or nails.

14. A method of dechlorinating skin, hair and/or nails of humans comprising contacting said skin, hair and/or nails with the aqueous composition of claim 6 for sufficient time to reduce halogen effects on the skin, hair and/or nails.

15. The method of claim 9 wherein efficiency of the method has results determined on human hair having an average thickness of from 50-100 micrometers is immersed in chlorinated water having 2 ppm free chlorine and less than 0.2 ppm combined chlorine for 15 minutes and this hair after immersion is then washed for 1.5-2.0 minutes with the aqueous composition and rinsed in tap water with scrubbing to remove at least 95% by weight of the applied composition, and then the rinsed hair is leached in 1.0 liters of deionized water for 2 minutes and the free chlorine level and the combined chlorine level is measured as the free chlorine level is below 0.1 ppm and the combined chlorine level is be below 0.05 ppm.

16. The method of claim 10 wherein efficiency of the method has results determined on human hair having an average thickness of from 50-100 micrometers is immersed in chlorinated water having 2 ppm free chlorine and less than 0.2 ppm combined chlorine for 15 minutes and this hair after immersion is then washed for 1.5-2.0 minutes with the aqueous composition and rinsed in tap water with scrubbing to remove at least 95% by weight of the applied composition, and then the rinsed hair is leached in 1.0 liters of deionized water for 2 minutes and the free chlorine level and the combined chlorine level is measured as the free chlorine level is below 0.1 ppm and the combined chlorine level is be below 0.05 ppm.

17. The method of claim 11 wherein efficiency the method has results determined on human hair having an average thickness of from 50-100 micrometers is immersed in chlorinated water having 2 ppm free chlorine and less than 0.2 ppm combined chlorine for 15 minutes and this hair after immersion is then washed for 1.5-2.0 minutes with the aqueous composition and rinsed in tap water with scrubbing to remove at least 95% by weight of the applied composition, and then the rinsed hair is leached in 1.0 liters of deionized water for 2 minutes and the free chlorine level and the combined chlorine level is measured as the free chlorine level is below 0.1 ppm and the combined chlorine level is be below 0.05 ppm.

18. The aqueous composition of claim 1 carried in an absorbent sheet in sufficient concentration of the aqueous composition on the absorbent sheet that pressure between the absorbent sheet and skin will transfer some of the aqueous composition onto the skin.

19. The aqueous composition of claim 2 wherein the the oil-soluble ascorbate is a bis or tri- or tetraalkanol ascorbate.

20. The method of claim 10 wherein the oil-soluble ascorbate is a bis or tri- or tetraalkanol ascorbate.
